(19) United States
(12) Patent Application Publication
(10) Pub. No.: US 2008/0119880 A1
(43) Pub. Date: May 22, 2008

(54) LAPAROSCOPIC SURGICAL CLAMP

(76) Inventor: David Z. J. Chu, San Gabriel, CA (US)

Correspondence Address: CHAN LAW GROUP LC, 1055 W. 7TH ST,, SUITE 1880, LOS ANGELES, CA, 90017, US

(21) Appl. No.: 11/560,750

(22) Filed: Nov. 16, 2006

(51) Int. Cl. A61B 17/08

(52) U.S. Cl. 606/157

(57) ABSTRACT

A laparoscopic surgical clamp for use in minimally invasive surgical procedures includes an elongated shaft, a first set and a second set of double jaws connected to distal end of shaft and moveable relative to one another, and a double handle assembly operatively connected to at least one of the double jaws. The double jaws include an opened position in which the double jaws are separated and substantially parallel to one another and a closed position in which the double jaws are adjacent and substantially parallel to one another. At least one articulation segment is located on each jaw such that the double jaws may be positioned in a U- or V-shaped configuration when in an articulated position. A plurality of fenestration is located on at least one jaw of the double jaws to allow suturing through the double jaws to secure the surgical clamp to the organ tissue.

### BACKGROUND OF THE INVENTION

1. Field of the Invention

The present invention relates to the field of medical devices, and more specifically to a laparoscopic surgical clamp for use in minimally invasive surgical procedures. The laparoscopic surgical clamp is particularly beneficial in that it allows for resections of liver lesions of different sizes and anatomical locations and lesions in close proximity to the major liver vessels.

2. Description of the Prior Art

Surgical clamps that use various types of clamping jaws and clamping configurations (i.e., curved, straight, and the like) are well known in the medical device art. In particular, large tissue clamps to accommodate substantial vessel or tissue compression for open surgical procedures such as laparotomies are used for major vascular procedures on the aorta or vena cava with Satinsky or Sehrt's clamps, and for gynecological procedures with Haney clamps, which also have a serrated mechanism at the clamp tip to prevent tissue slippage. Gastric clamps such as the Payrs clamp combines clamping and crushing of tissues for ease of transection. Large liver clamps of the Lin type have been used for hemostatic purposes. The Chu liver clamp was designed as a Lin liver clamp except it has larger and curved jaws.

For laparoscopic approaches, examples of vascular clamps that have been developed are disclosed in Anderson, et al., Patent Publication No. 2005/0251184, Douglas, et al., Publication No. 2005/0165429, Gold, et al., Publication No. 2003/0212435, and Schwarz, et al., Patent Publication No. 2005/0147585. Similarly, examples of sutures and suture methods that have been developed for use in minimally invasive surgical procedures are disclosed in Nguyen, et al., Patent Publication No. 2003/0191481, and Nobles, et al., U.S. Pat. No. 6,911,034.

In the minimally invasive setting, peripheral liver masses are currently tackled by the hand assistance method and other liver parenchyma transection techniques. The more centrally located liver nodules and masses present a unique challenge and most are resected using open surgical techniques. With the introduction of minimally invasive surgical procedures in recent years, surgical sites have decreased in size, and obstruction by large, surgical clamps of the surgeon's field of visibility or access to other locations at the surgical site have become problematic. Furthermore, while open liver resections have several techniques to control parenchymal bleeding from the liver, laparoscopic liver resections are primarily limited by the inability to control bleeding.

In addition, liver ligatures are difficult to apply by laparoscopic means. Liver sutures by their very nature are required to be large in terms of tissue bite for both vascular and bile duct control and hepatic parenchymal compression. In the open surgical procedure, the needles and sutures are ideally 1-2 inches in diameter and the caliber of sutures are either 0 or #1. Sutures of this size are difficult to introduce and maneuver within the minimally invasive body cavity.

It would be desirable to have a low-profile laparoscopic surgical clamp design that can be introduced into the abdominal cavity through a small port for use in minimally invasive surgical procedures. It would be also desirable to have a low-profile laparoscopic surgical clamp that can be applied to a patient's internal organs, such as the liver, lung, kidney, and spleen, during minimally invasive surgical procedures. It would be further desirable to have a laparoscopic surgical clamp that will allow resections of liver lesions of different sizes and anatomical locations and lesions in close proximity to the major liver vessels.

### SUMMARY OF THE INVENTION

The present invention provides a laparoscopic surgical clamp for use in minimally invasive surgical procedures. The laparoscopic surgical clamp may be applied to various internal organs of a patient, including the liver, lung, spleen, and kidney. In a first aspect, the present invention includes an elongated shaft having a proximal end and a distal end. A first set and a second set of double jaws are connected to the distal end of the elongated shaft and moveable relative to one another. The first set and second set of double jaws include an opened position in which the first set and second set of double jaws are separated and substantially parallel to one another and a closed position in which the first set and second set of double jaws are adjacent and substantially parallel to one another. The first set and second set of double jaws may assume a curved configuration along the axial lengths thereof to accommodate the curvature of the patient's organ tissue.

The laparoscopic surgical clamp is narrow and elongated when the first set and second set of double jaws are in the closed position. The first set and second set of double jaws extend laterally from the elongated shaft when in the closed position. A removable insert may be positioned on an inside surface of each of the first set and second set of double jaws allowing cushioning of a patient's clamped tissue.

At least one articulation segment is located on each jaw of the first set and second set of double jaws such that the jaws may be positioned in either a U- or V-shaped configuration when in an articulated position. The first set and second set of double jaws are not substantially parallel to each other in the articulated position. The first set and second set of double jaws extend distally relative to the elongated shaft in the articulated position.

A plurality of fenestration is located on at least one jaw of the first set and second set of double jaws allowing suturing through the first set and second set of double jaws. The suturing through the fenestration of the first set and second set of double jaws secures the surgical clamp to a patient's clamped tissue during the minimally invasive surgical procedure. At least one clip is applied across the suturing to secure the first set and second set of double jaws to the patient's clamped tissue.

The laparoscopic surgical clamp further includes a double hand assembly operatively connected to at least one of the first set and second set of double jaws such that the jaws may move between the opened and closed positions relative to the motion of the double hand assembly. The double handle assembly has a locking mechanism allowing ease of engagement of the first set and second set of double jaws between the opened and closed positions. The double handle assembly has an inner handle allowing control of the first set of double jaws into the closed position.

The first set and second set of double jaws are controlled by manipulating a plurality of fine cables having a tension and extending distally from the double handle assembly and connected to the first set and second set of double jaws. At least one of the plurality of cables is connected to the articulations of the first set and second set of double jaws to effect closure or articulation of the first set and second set of double jaws. The tension of the plurality of cables is operatively controlled by the double handle assembly. The first set and second set of double jaws are capable of being changed to at least one orientation with respect to the elongated shaft connecting the jaws to the double handle assembly.

The laparoscopic surgical clamp is capable of accessing an abdominal cavity through a 15 mm port. The first set and second set of double jaws have an operative length of about 12-15 cm. The laparoscopic surgical clamp, including the first set and second set of double jaws, the elongated shaft, and the double hand assembly, has an operative length of about 37-50 cm.

In another aspect of the present invention, the laparoscopic surgical clamp includes an elongated shaft having a proximal end and a distal end. A first set and second set of double jaws are connected to the distal end of the elongated shaft and moveable relative to one another. The first set and second set of double jaws include an opened position in which the first set and second set of double jaws are separated and substantially parallel to one another and a closed position in which the first set and second set of double jaws are adjacent and substantially parallel to one another.

At least one articulation segment is located on each jaw of the first set and second set of double jaws such that the jaws may be positioned in either a U- or V-shaped configuration when in an articulated position. A plurality of fenestration is located on at least one jaw of the first set and second set of double jaws allowing suturing through the first set and second set of double jaws.

The laparoscopic surgical clamp further includes a double hand assembly having an inner handle portion operatively connected to at least one of the first set and second set of double jaws such that the jaws may move between the opened and closed positions relative to the motion of the double hand assembly. The double handle assembly has a switch-type locking mechanism configured on an outside surface thereof allowing ease of engagement of the first set and second set of double jaws between the opened and closed positions.

In a further aspect of the present invention, the laparoscopic surgical clamp includes an elongated shaft having a proximal end and a distal end. A first set and second set of double jaws are connected to the distal end of the elongated shaft by a pivot and are pivotally moveable relative to one another. The first set and second set of double jaws include an opened position in which the first set and second set of double jaws are separated and substantially parallel to one another and a closed position in which the first set and second set of double jaws are adjacent and substantially parallel to one another.

At least one articulation segment is located on each jaw of the first set and second set of double jaws such that the jaws may be positioned in either a U- or V-shaped configuration when in an articulated position. A plurality of fenestration is located on at least one jaw of the first set and second set of double jaws allowing suturing through the first set and second set of double jaws for securing the jaws to a patient's clamped tissue. At least one clip is applied across the suturing for securing the first and second set of double jaws to the patient's clamped tissue.

The laparoscopic surgical clamp further includes a double hand assembly having an inner handle portion operatively connected to at least one of the first set and second set of double jaws by a fine cable such that the jaws may move between the opened and closed positions relative to the motion of the double hand assembly. The double handle assembly has a locking mechanism configured on an outside surface thereof allowing ease of engagement of the first set and second set of double jaws between the opened and closed positions.

These and other features and advantages of this invention will become further apparent from the detailed description and accompanying figures that follow. In the figures and description, numerals indicate the various features of the disclosure, like numerals referring to like features throughout both the drawings and the description.

### BRIEF DESCRIPTION OF THE DRAWINGS

FIG. 1 is a perspective view of a laparoscopic surgical clamp according to the present invention.

FIG. 2 is a cross-sectional view of the laparoscopic surgical clamp taken along lines 2-2 of FIG. 1.

FIG. 3 is an enlarged perspective view of a first set and a second set double jaw of the laparoscopic surgical clamp of FIG. 1.

FIG. 4 is an enlarged perspective view of the first double jaw of the laparoscopic surgical clamp of FIG. 1 having spaces within the jaws for placement of sutures.

FIG. 5 is an enlarged perspective view of a double handle assembly of the laparoscopic surgical clamp of FIG. 1.

FIG. 6 is a partial perspective view of a cable system for controlling the jaws of the laparoscopic surgical clamp of FIG. 1.

FIG. 7 is an enlarged partial perspective view of a distal end of exemplary sutures that may be used in combination with the laparoscopic surgical clamp of FIG. 1 according to the present invention.

FIG. 8A is a perspective side view of the laparoscopic surgical clamp in place during resection of the liver according to the present invention.

FIG. 8B is a perspective side view of the suture and clips in place during post-resection of the liver according to the present invention.

### DETAILED DESCRIPTION OF THE PREFERRED EMBODIMENTS

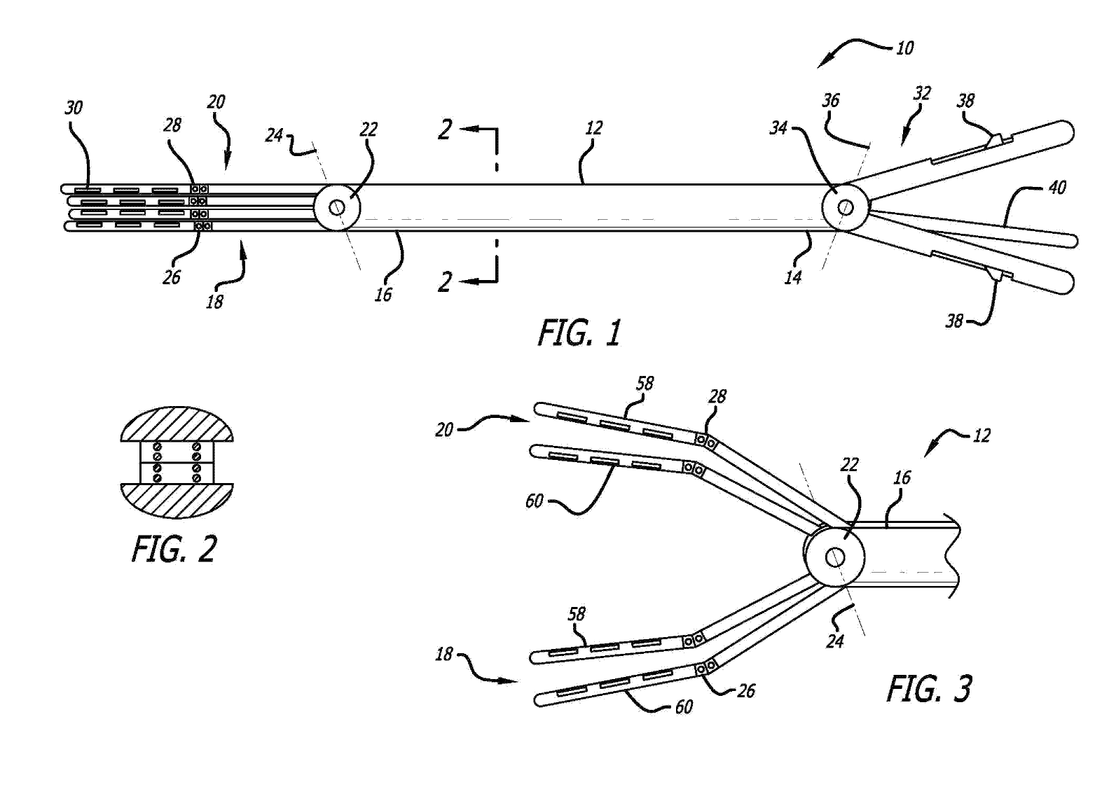

The laparoscopic surgical clamp 10 of the present invention is generally illustrated in FIG. 1. It is contemplated by the present invention that the laparoscopic surgical clamp 10 is particularly configured to be applied to a patient's internal organs, including, but not limited to, the liver, lung, spleen and kidney, during minimally invasive surgical procedures. The laparoscopic surgical clamp 10 includes an elongated shaft 12 having a proximal end 14 and a distal end 16. First set and second set of double jaws 18, 20 are connected to the distal end 16 of the elongated shaft 12 by a pivot 22 and are pivotally moveable relative to one another about a rotational axis 24.

The laparoscopic surgical clamp has the capability of being manipulated into various positions as desired by the surgeon during the minimally invasive surgical procedure. In an opened position, the first set and second set of double jaws 18, 20 are separated and substantially parallel to one another. In a closed position (FIG. 1), the first set and second set of double jaws 18, 20 are adjacent and substantially parallel to one another. In an articulated position (FIG. 3), at least one articulation segment 26, 28 on each jaw of the first set and second set of double jaws 18, 20 is configured such that the jaws may be positioned in a U- or V-shaped configuration. When the first set and second set of double jaws 18, 20 are in the articulated position (FIG. 3), the jaws 18, 20 are not substantially parallel to one another and extend distally relative to the elongated shaft 12.

Referring further to FIG. 1, a plurality of fenestration 30 is formed on at least one jaw of the first set and second set of double jaws 18, 20 of the laparoscopic surgical clamp 10. This allows the surgeon to thread suturing (FIG. 8A) through the first set and second set of double jaws 18, 20 to firmly secure the surgical clamp to a patient's clamped tissue during the minimally invasive surgical procedure.

The laparoscopic surgical clamp further includes a double handle assembly 32 operatively connected to at least one of the first set and second set of double jaws 18, 20 by a fine cable 44 (FIG. 6) such that the jaws 18, 20 may move between the opened and closed positions with ease relative to the motion of the double handle assembly 32. The double handle assembly 32 is connected to the elongated shaft 12 by a pivot 34 and is pivotally movable about a rotational axis 36. The double handle assembly 32 may be connected to the shaft by means other than a pivot that are known in the art.

In one embodiment of the present invention, the double handle assembly is configured to have a switch-type locking mechanism 38, which allows ease of engagement of the first set and second set of double jaws 18, 20 between the opened and closed positions. The double handle assembly 32 also includes an inner handle 40 allowing control of the first set of double jaws 18, 20 into the closed position. The first set and second set of double jaws 18, 20 are capable of being changed to at least one orientation with respect to the shaft 12 operatively connecting the jaws 18, 20 to the double handle assembly 32.

The double handle assembly 32 allows the surgeon to hold the laparoscopic surgical clamp 10 with one hand, open and close the first set and second set of double jaws 18, 20, and apply the and locking mechanism 38. The laparoscopic surgical clamp 10 will remain locked as long as the surgeon desires and can be unlocked by the surgeon with ease to readjust position of surgical clamp 10 or remove the surgical clamp 10 from the body cavity. In addition, the pressure applied to the liver parenchyma or other organ parenchyma through the jaws 18, 20 can be adjustable. In the locked position, the double handle assembly 32 will need to be protected and avoid movement to prevent the jaws 18, 20 of the laparoscopic surgical clamp 10 on the solid organ from moving or torquing into undesirable positions. The laparoscopic surgical clamp 10 when properly applied will compress the liver to the point of achieving hemostasis and not cut into liver parenchyma or other organ parenchyma. Tears into the solid organ parenchyma can still occur.

FIG. 2 illustrates a cross-sectional view of the laparoscopic surgical clamp of FIG. 1. The laparoscopic surgical clamp is narrow and elongated when the first set and second set of double jaws are in the closed position (FIG. 1). The first set and second set of double jaws 18, 20 extend laterally from the elongated shaft when in the closed position (FIG. 1).

In FIG. 3, the first set and second set of double jaws 18, 20 of the laparoscopic surgical clamp 10 of FIG. 1 are illustrated in greater detail. The articulation segments 26, 28 of the first set and second set of double jaws 18, 20 are configured such that the jaws may be positioned in a U- or V-shaped configuration.

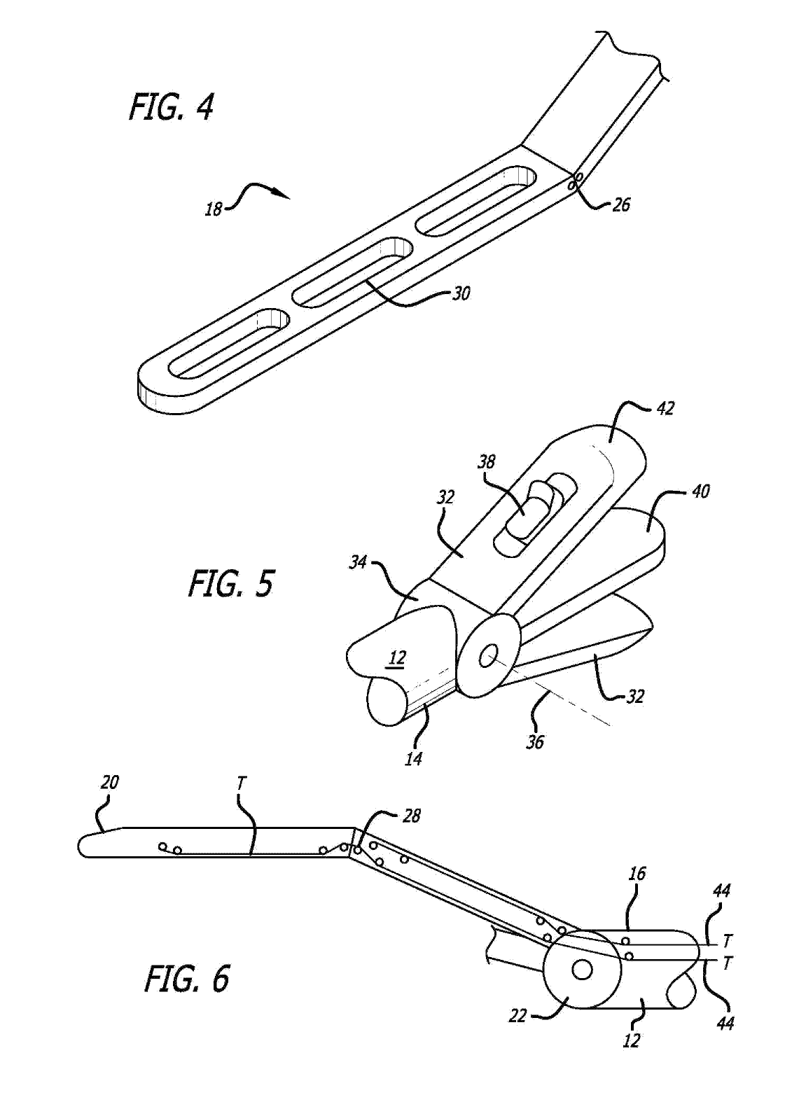

Referring now to FIG. 4, a jaw portion 18 of the first set of double jaws 18 of FIG. 1 is shown in greater detail. The jaw portion includes a plurality of fenestration 30 or open spaces configured into its design such that the surgeon can pass sutures therethrough to secure the surgical clamp to the patient's clamped tissue during the minimally invasive surgical procedure.

FIG. 5 illustrates an enlarged perspective view of the double handle assembly 32 of the laparoscopic surgical clamp 10 of FIG. 1. In one embodiment, the double handle assembly 32 includes a switch-type locking mechanism 38 configured into its design on an outside surface 42 thereof. The locking mechanism 38 is not limited to a switch-type design as other types of locking mechanisms, such as a lever (not shown), may be used in accordance with the present invention. As previously set forth, the locking mechanism 38 allows ease of engagement of the first set and second set of double jaws 18, 20 between the opened and closed positions. The inner handle 40 allows control of the first set of double jaws 18, 20 into the closed position.

Referring now to FIG. 6, a partial perspective view of a cable system for controlling the jaws 18, 20 of the laparoscopic surgical clamp of FIG. 1 is illustrated in detail. Control of the first set and second set of double jaws 18, 20 is manipulated by a plurality of fine cables 44 having a tension T. Tension of the fine cables 44 is operatively controlled by the double handle assembly 32. Tension applied to the fine cables 44 and the ability to control tension on the cables by the locking mechanism 38 will determine the closure pressure on the jaws 18, 20, which in turn determines the tissue pressure resulting in the desired hemostasis. The pressure can be visually gauged by the surgeon as excessive pressure can cause tissue fracture.

In one embodiment, at least one of the plurality of fine cables 44 is connected to the articulated segments 26, 28 of the first set and second set of double jaws 18, 20 to effect both closure and articulation of the jaws 18, 20. Tension is transmitted to the jaws 18, 20 through the plurality of fine cables 44 by applying greater closing or apposing pressure on the double handle assembly 32. The locking mechanism 38 on the double handle assembly 32 is configured to lock in the applied pressure.

The first set and second set of double jaws 18, 20 of the laparoscopic surgical clamp may be aligned, shaped and/or sized according to their intended use depending on such factors as the organ or tissue clamped, surgeon preference, type of surgical procedure involved, and the like. For example, the first set and second set of double jaws 18, 20 may be straight or curved along an axial length thereof to accommodate the curvature of the patient's organ tissue, and long or short. The jaws may be designed such that when directly opposed, they are transversely, horizontally, or vertically aligned with each other. The double row design of the jaws 18, 20 provides the laparoscopic surgical clamp with greater compressibility of the clamped tissue. In further embodiments, it is contemplated by the present invention that depending on the material composition of the first set and second set of double jaws 18, 20, they may be designed to have varying degrees of flexibility and stiffness along their axial lengths thereof such that they are malleable at their distal ends and can be shaped by the surgeon prior to deployment of the laparoscopic surgical clamp.

Once the first set and second set of double jaws 18, 20 are applied and liver resection has begun, the jaws 18, 20 of the laparoscopic surgical clamp 10 can slip off the edge of the liver. Thus, suturing may be necessary to avoid slippage of the first set and second set of double jaws 18, 20. Two or more laparoscopic surgical clamps 10 may be necessary to complete hemostasis on the liver to allow resection with minimal blood loss. For example, the application of each surgical clamp 10 isolates the blood supply to the segment occupied by the tumor and the two surgical clamps 10 apposed in a triangular manner isolate a pie shape volume for complete hemostasis. Once the surgical clamp is applied, further compression may be achieved by suturing the jaws 18, 20 closer together to prevent jaw slippage off cut edge of liver or other organs during the minimally invasive surgical procedure.

Two laparoscopic surgical clamps 10 can be used during the minimally invasive surgical procedure in which the surgical clamps 10 are applied from opposing angles for central lesions and large tumors. The laparoscopic surgical clamp 10 of the present invention enables the surgeon to conduct resections of liver lesions of differing sizes and anatomical locations and lesions closer to the major liver vessels. The laparoscopic surgical clamp 10 can also be used on the spleen in a laparoscopic partial splenectomy, or on the kidney, in a laparoscopic partial nephrectomy.

It is contemplated by the present invention that the double jaws 18, 20 can form a U- or V-shaped configuration as earlier disclosed herein. The U-shaped configuration may be further achieved by using two surgical clamps and applying the two jaws 18, 20 at varying angles of deployment. The angling of the jaws 18, 20 requires an additional cable system (not shown) at 90° from the direction of fine cables that will close the jaws 18, 20. The V-shaped configuration may be further achieved by using two surgical clamps 10 coming from two separate trochars (not shown) and converging at a point on the liver parenchyma thereby isolating a V or pie-shape of the liver to be resected.

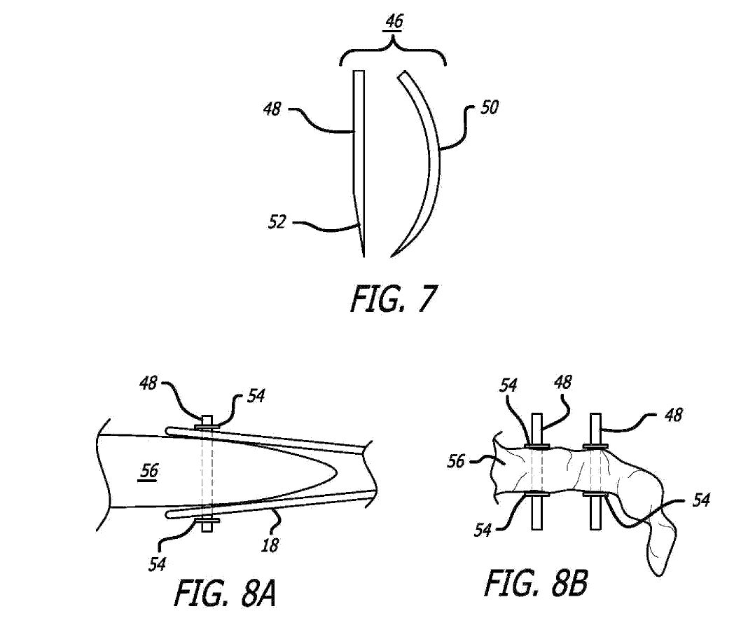

Referring now to FIG. 7 is an enlarged partial perspective view of a distal end of exemplary sutures 46, such as straight or curved, which may be used in conjunction with the laparoscopic surgical clamp of FIG. 1. The straight suture 48 design can be punctured through the entire thickness of the liver tissue with ease. The curved suture 50 design can be used to apply the suture through the same surface of the liver or other organ and close or compress a liver lesion or other organ tear or appose two livers or other organ surfaces.

In one embodiment, the suture 48 is hollow as a thin straw with a pointed distal end 52 to allow puncturing through the liver or other solid organ parenchyma. The suture 48 can be pulled into the body cavity with ease using a laparoscopic grasper. The suture 48 length is variable and can be trimmed to a desired length depending on the particular minimally invasive surgical procedure. The distal suture tip 52 is narrow and solid as in a pointed tip to allow introduction into the liver or other solid organs such as the spleen or kidney. The material of the suture 48 is bio-absorbable, such as a polyglycolic polymer, and is about 1.5-2 mm in diameter and about 10 cm long with a needle point 52.

The suture 48 can be passed through the plurality of fenestration 30 of at least one of the first set and second set of double jaws 18, 20 of the laparoscopic surgical clamp 10 of the present invention. The suture 48 can be clipped 54 by a metal clip applicator and will compress the round hollow suture flat, which will increase the surface securing the clip 54 at the level desired. Further compression may be achieved by reapplying the clip 54 and cutting off the excess length and discarding the previous clip. This maneuver may be applied to both ends of the suture 48 securing the liver or other organ parenchyma into a compressed position. Once the resection is complete, additional conventional sutures 48 or clips 54 can be applied on the cut end of the vessels and small bile ducts. Once the vascular control of the liver or other organ is satisfactory the laparoscopic surgical clamp 10 can be disengaged.

FIG. 8A illustrates the laparoscopic surgical clamp 10 in place secured with clips 54 during resection of the liver 56 according to the present invention. FIG. 8B illustrates the suture 48 and clips 54 in place compressing the liver 56 with the surgical clamp 10 removed during post-resection of the liver 56.

The laparoscopic surgical clamp is uniquely designed to have a low-profile such that it is capable of accessing an abdominal cavity through a 15 mm port during the minimally invasive surgical procedure. In one embodiment, the first set and second set of double jaws 18, 20 may have an operative length of about 12-15 cm. The laparoscopic surgical clamp 10, including the first set and second set of double jaws 18, 20, elongated shaft 12, and the double handle assembly 32, may have an overall operative length of about 37-50 cm.

While the laparoscopic surgical clamp 10 is particularly contemplated for use in laparoscopic or endoscopic minimally invasive surgical procedures, the present invention may also be used in a variety of open surgical procedures, including those requiring occlusion of organs such as the liver, lung, spleen, kidney, and the like.

The laparoscopic surgical clamp 10 may be fabricated from virtually any biocompatible material, including, but not limited to, stainless steel and its alloys, titanium alloys (i.e., nickel-titanium), polymers (i.e., polyethylene and copolymers thereof, and the like), and combinations thereof.

In a further embodiment, the first set and second set of double jaws 18, 20 may include a removable insert (not shown) on the inside surface of each jaw 18, 20 to cushion the patient's clamped tissue. This prevents the patient's tissue from being damaged or otherwise torn by the first set and second set of double jaws 18, 20 of the laparoscopic surgical clamp during the minimally invasive surgical procedure. The cushion may be formed from various materials known in the art that are compliant and hence will provide a cushioning effect to the clamped tissue, such as those described in U.S. application Ser. No. 10/783,811, filed Feb. 19, 2004, which is incorporated herein by reference in its entirety. The cushion inserts may be attached to the jaws 18, 20 by techniques known in the art, such as those described in the immediately aforementioned U.S. application.

The laparoscopic surgical clamp 10 of the present invention may be either disposable or reusable (i.e., sterilizable). Alternatively, portions of the laparoscopic surgical clamp 10 may be reused and sterilized for subsequent use and other portions may be disposed of for no further use.

Having now described the invention in accordance with the requirements of the patent statutes, those skilled in the art will understand how to make changes and modifications in the present invention to meet their specific requirements or conditions. Such changes and modifications may be made without departing from the scope and spirit of the invention as set forth in the following claims.

What is claimed is:

1. A laparoscopic surgical clamp for use in minimally invasive surgical procedures, comprising:
an elongated shaft having a proximal end and a distal end;
a first set and a second set of double jaws connected to the distal end of the elongated shaft and moveable relative to one another, the first set and second set of double jaws comprising:
an opened position wherein the first set and second set of double jaws are separated and substantially parallel to one another and a closed position wherein the first set and second set of double jaws are adjacent and substantially parallel to one another;
at least one articulation segment on each jaw of the first set and second set of double jaws such that the first set and second set of double jaws may be positioned in a U- or V-shaped configuration when in an articulated position;
a plurality of fenestration on at least one jaw of the first set and second set of double jaws allowing suturing through the first set and second set of double jaws;
a double handle assembly operatively connected to at least one of the first set and second set of double jaws such that the jaws may move between the opened and closed positions relative to the motion of the double handle assembly;
the double handle assembly having a locking mechanism allowing ease of engagement of the first set and second set of double jaws between the opened and closed positions.

2. The laparoscopic surgical clamp of claim 1, wherein the first set and second set of double jaws are connected to the shaft by a pivot and are pivotally moveable relative to one another.

3. The laparoscopic surgical clamp of claim 1, wherein the first set and second set of double jaws are in an articulated position and not substantially parallel to one another.

4. The laparoscopic surgical clamp of claim 1, wherein the surgical clamp may be applied to internal organs of a patient including at least one of a liver, lung, spleen, and kidney.

5. The laparoscopic surgical clamp of claim 1, wherein the surgical clamp is narrow and elongated when the first set and second set of double jaws are in the closed position.

6. The laparoscopic surgical clamp of claim 1, wherein the first set and second set of double jaws extend laterally from the elongated shaft when in the closed position.

7. The laparoscopic surgical clamp of claim 1, wherein the first set and second set of double jaws extend distally relative to the elongated shaft when in the articulated position.

8. The laparoscopic surgical clamp of claim 1, wherein the first set and second set of double jaws may include a removable insert on an inside surface of each jaw allowing cushioning of a patient's clamped tissue.

9. The laparoscopic surgical clamp of claim 1, wherein the laparoscopic surgical clamp is capable of accessing an abdominal cavity through a 15 mm port.

10. The laparoscopic surgical clamp of claim 1, wherein control of the first set and second set of double jaws is manipulated by a plurality of fine cables having a tension and extending distally from the double handle assembly and connected to the first set and second set of double jaws.

11. The laparoscopic surgical clamp of claim 10, wherein at least one of the plurality of cables is connected to the articulated segments of the first set and second set of double jaws to effect closure of the first set and second set of double jaws.

12. The laparoscopic surgical clamp of claim 10, wherein at least one of the plurality of cables is connected to the articulated segments of the first set and second set of double jaws to effect articulation of the first set and second set of double jaws.

13. The laparoscopic surgical clamp of claim 10, wherein the tension of the plurality of cables is operatively controlled by the double handle assembly.

14. The laparoscopic surgical clamp of claim 1, wherein the first set and second set of double jaws are capable of being changed to at least one orientation with respect to the shaft connecting the jaws to the double handle assembly.

15. The laparoscopic surgical clamp of claim 1, wherein the suturing through the fenestration of the first set and second set of double jaws secures the surgical clamp to a patient's clamped tissue during the minimally invasive surgical procedure.

16. The laparoscopic surgical clamp of claim 15, wherein at least one clip is applied across the suturing to secure the first set and second set of double jaws to the patient's clamped tissue during the minimally invasive surgical procedure.

17. The laparoscopic surgical clamp of claim 1, wherein the first set and second set of double jaws may assume a curved configuration along the axial lengths thereof to accommodate the curvature of a patient's organ tissue.

18. The laparoscopic surgical clamp of claim 1, wherein the first set and second set of double jaws have an operative length of about 12-15 cm.

19. The laparoscopic surgical clamp of claim 1, wherein the surgical clamp, including the first set and second set of double jaws, the elongated shaft, and the double handle assembly, has an operative length of about 37-50 cm.

20. The laparoscopic surgical clamp of claim 1, wherein the double handle assembly includes an inner handle allowing control of the first set of double jaws into the closed position.

21. The laparoscopic surgical clamp of claim 1, wherein the double handle assembly is operatively connected to at least one of the first set and second set of double jaws by a fine cable.

22. The laparoscopic surgical clamp of claim 1, wherein the double handle assembly is connected to the shaft by a pivot such that the double handle assembly is pivotally moveable about a rotational axis.

23. The laparoscopic surgical clamp of claim 1, wherein the suturing prevents the surgical clamp from slipping off a cut edge of organ tissue.

24. The laparoscopic surgical clamp of claim 1, wherein the first set and second set of double jaws are malleable at a distal end such that the jaws can be shaped by a surgeon prior to deployment of the surgical clamp.

25. A laparoscopic surgical clamp for use in minimally invasive surgical procedures, comprising:
an elongated shaft having a proximal end and a distal end;
a first set and a second set of double jaws connected to the distal end of the elongated shaft and moveable relative to one another, the first set and second set of double jaws comprising:
an opened position wherein the first set and second set of double jaws are separated and substantially parallel to one another and a closed position wherein the first set and second set of double jaws are adjacent and substantially parallel to one another;
at least one articulation segment on each jaw of the first set and second set of double jaws such that the first set and second set of double jaws may be positioned in a U- or V-shaped configuration when in an articulated position;
a plurality of fenestration on at least one jaw of the first set and second set of double jaws to allow suturing through the first set and second set of double jaws;
a double handle assembly having an inner handle portion operatively connected to at least one of the first set and second set of double jaws such that the jaws may move between the opened and closed positions relative to the motion of the double handle assembly;
the double handle assembly having a switch-type locking mechanism configured on an outside surface thereof allowing ease of engagement of the first set and second set of double jaws between the opened and closed positions.

26. A laparoscopic surgical clamp system for use in minimally invasive surgical procedures, comprising:
an elongated shaft having a proximal end and a distal end;
a first set and a second set of double jaws connected to the distal end of the elongated shaft by a pivot and are pivotally moveable relative to one another, the first set and second set of double jaws comprising:
an opened position wherein the first set and second set of double jaws are separated and substantially parallel to one another and a closed position wherein the first set and second set of double jaws are adjacent and substantially parallel to one another;
at least one articulation segment on each jaw of the first set and second set of double jaws such that the first set and second set of double jaws may be positioned in a U- or V-shaped configuration when in an articulated position;
a plurality of fenestration on at least one jaw of the first set and second set of double jaws allowing suturing through the first set and second set of double jaws for securing the jaws to a patient's clamped tissue;
wherein at least one clip is applied across the suturing for securing the first and second set of double jaws to the patient's clamped tissue;
a double handle assembly having an inner handle portion operatively connected to at least one of the first set and second set of double jaws by a fine cable such that the jaws may move between the opened and closed positions relative to the motion of the double handle assembly;
the double handle assembly having a locking mechanism configured on an outside surface thereof allowing ease of engagement of the first set and second set of double jaws between the opened and closed positions.
